SEQUENCE LISTING (20 sequences)

SEQ ID NO: 1 - DNA, 336 bp, Artificial Sequence
  Constructed Sequence
atggctctgtggatgagactgctgccactgctggctctgctcgcactgtgggctccagctcctacaagagccttcgtgaaccagcacctgtgcggctctcatctggtggaagccctgtatctcgtgtgcggcgagaggggctttttctacacccctcgggccaagagagaggtcgaggatctgcaagtgcgcgacgttgaactggctggtgctcctggcgaaggtggactgcaacctctggctctggaaggcgccagacagaaaaggggcatcgtggaacagtgctgcaccagcatctgcagcctgtaccagctggaaaactactgcaactgatga

SEQ ID NO: 2 - protein, 110 aa, Artificial Sequence
  Constructed sequence
MALWMRLLPLLALLALWAPAPTRAFVNQHLCGSHLVEALYLVCGERGFFYTPRAKREVEDLQVRDVELAGAPGEGGLQPLALEGARQKRGIVEQCCTSICSLYQLENYCN

SEQ ID NO: 3 - DNA, 324 bp, Artificial Sequence
  Constructed sequence
atgtacaagatgcagctgctgagctgtatcgccctgacactggtgctggtggccaacagcttcgtgaaccagcacctgtgcggaagccatctggtggaagccctgtatctcgtgtgtggcgagaggggcttcttctacacccctcgggccaagagagaggtcgaggatctgcaagtgcgcgacgttgaactggctggtgctcctggcgaaggtggactgcaacctctggctctggaaggcgccagacagaaaaggggcatcgtggaacagtgctgcaccagcatctgcagcctgtaccagctggaaaactactgcaactgatga

SEQ ID NO: 4 - protein, 106 aa, Artificial Sequence
  Constructed sequence
MYKMQLLSCIALTLVLVANSFVNQHLCGSHLVEALYLVCGERGFFYTPRAKREVEDLQVRDVELAGAPGEGGLQPLALEGARQKRGIVEQCCTSICSLYQLENYCN

SEQ ID NO: 5 - DNA, 130 bp, adeno-associated virus 2
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 6 - DNA, 382 bp, Artificial Sequence
  CMV IE promoter
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattac

SEQ ID NO: 7 - DNA, 282 bp, Artificial Sequence
  CB promoter
tggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgg

SEQ ID NO: 8 - DNA, 973 bp, Artificial Sequence
  CBA intron
gtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaag

SEQ ID NO: 9 - DNA, 127 bp, Artificial Sequence
  Rabbit beta globin poly A
gatctttttccctctgccaaaaattatggggacatcatgaagccccttgagcatctgacttctggctaataaaggaaatttattttcattgcaatagtgtgttggaattttttgtgtctctcactcg

SEQ ID NO: 10 - DNA, 130 bp, adeno-associated virus 2
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 11 - DNA, 2679 bp, Artificial Sequence
  Constructed sequence
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgctagcgccaccatggctctgtggatgagactgctgccactgctggctctgctcgcactgtgggctccagctcctacaagagccttcgtgaaccagcacctgtgcggctctcatctggtggaagccctgtatctcgtgtgcggcgagaggggctttttctacacccctcgggccagaagagaggtcgaggatctgcaagtgcgcgacgttgaactggctggtgctcctggcgaaggtggactgcaacctctggctctggaaggcgccagacagaaaaggggcatcgtggaacagtgctgcaccagcatctgcagcctgtaccagctggaaaactactgcaactgatgagacctctagagcggccgcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 12 - DNA, 2667 bp, Artificial Sequence
  Constructed sequence
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgctagcgccaccatgtacaagatgcagctgctgagctgtatcgccctgacactggtgctggtggccaacagcttcgtgaaccagcacctgtgcggaagccatctggtggaagccctgtatctcgtgtgtggcgagaggggcttcttctacacccctcgggccaagagagaggtcgaggatctgcaagtgcgcgacgttgaactggctggtgctcctggcgaaggtggactgcaacctctggctctggaaggcgccagacagaaaaggggcatcgtggaacagtgctgcaccagcatctgcagcctgtaccagctggaaaactactgcaactgatgagacctctagagcggccgcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 13 - DNA, 336 bp, Canis familiaris
atggccctgtggatgaggctgctgcctctgctggccctgctggccctgtgggcccctgcccctaccagggccttcgtgaaccagcacctgtgcggcagccacctggtggaggccctgtacctggtgtgcggcgagaggggcttcttctacacccctaaggccaggaggaagagggaggacctgcaggtgagggacgtggagctggccggcgcccctggcgagggcggcctgcagcctctggccctggagggcgccaggaggaagaggggcatcgtggagcagtgctgcaccagcatctgcagcctgtaccagctggagaactactgcaactgatga

SEQ ID NO: 14 - protein, 110 aa, Canis familiaris
MALWMRLLPLLALLALWAPAPTRAFVNQHLCGSHLVEALYLVCGERGFFYTPKARRKREDLQVRDVELAGAPGEGGLQPLALEGARRKRGIVEQCCTSICSLYQLENYCN

SEQ ID NO: 15 - DNA, 2679 bp, Artificial Sequence
  Constructed sequence
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgctagcgccaccatggccctgtggatgaggctgctgcctctgctggccctgctggccctgtgggcccctgcccctaccagggccttcgtgaaccagcacctgtgcggcagccacctggtggaggccctgtacctggtgtgcggcgagaggggcttcttctacacccctaaggccaggaggaagagggaggacctgcaggtgagggacgtggagctggccggcgcccctggcgagggcggcctgcagcctctggccctggagggcgccaggaggaagaggggcatcgtggagcagtgctgcaccagcatctgcagcctgtaccagctggagaactactgcaactgatgagacctctagagcggccgcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 16 - protein, 20 aa, Canis familiaris
MYKMQLLSCIALTLVLVANS

SEQ ID NO: 17 - protein, 24 aa, Canis familiaris
MALWMRLLPLLALLALWAPAPTRA

SEQ ID NO: 18 - protein, 86 aa, Artificial Sequence
  Constructed sequence
FVNQHLCGSHLVEALYLVCGERGFFYTPRAKREVEDLQVRDVELAGAPGEGGLQPLALEGARQKRGIVEQCCTSICSLYQLENYCN

SEQ ID NO: 19 - protein, 86 aa, Canis familiaris
FVNQHLCGSHLVEALYLVCGERGFFYTPKARRKREDLQVRDVELAGAPGEGGLQPLALEGARRKRGIVEQCCTSICSLYQLENYCN

SEQ ID NO: 20 - protein, 106 aa, Artificial Sequence
  Constructed sequence
MYKMQLLSCIALTLVLVANSFVNQHLCGSHLVEALYLVCGERGFFYTPKARRKREDLQVRDVELAGAPGEGGLQPLALEGARRKRGIVEQCCTSICSLYQLENYCN

(19) United States
(12) Patent Application Publication
(10) Pub. No.: US 2024/0010699 A1
(43) Pub. Date: Jan. 11, 2024

(54) VIRAL VECTORS ENCODING CANINE INSULIN FOR TREATMENT OF METABOLIC DISEASES IN DOGS

(71) Applicant: The Trustees of the University of Pennsylvania, Philadelphia, PA (US)

(72) Inventors: James M. Wilson, Philadelphia, PA (US); Christian Hinderer, Baltimore, MD (US); Makoto Horiuchi, Wallingford, PA (US)

(73) Assignee: The Trustees of the University of Pennsylvania, Philadelphia, PA (US)

(21) Appl. No.: 18/250,471

(22) PCT Filed: Nov. 4, 2021
(86) PCT No.: PCT/US2021/058105

§ 371 Date: Apr. 25, 2023

Related U.S. Application Data
(60) Provisional application No. 63/109,620, filed on Nov. 4, 2020.

(51) Int. Cl. C07K 14/62; C12N 15/86; A61P 3/10; A61K 48/00

(52) U.S. Cl. CPC C07K 14/62; C12N 15/86; A61P 3/10; A61K 48/005

(57) ABSTRACT

Compositions and methods for treating diabetes in a canine are provided. A viral vector is provided which includes a nucleic acid molecule comprising a sequence encoding a canine insulin polypeptide.

### BACKGROUND OF THE INVENTION

One in 500 dogs in the U.S have a condition similar to diabetes in humans. Though there are no universally accepted definitions of dog diabetes, three forms have been described: Type I diabetes mellitus (T1DM), type II diabetes mellitus (T2DM), and type III diabetes mellitus (T3DM).

Type I diabetes mellitus (sometimes also called insulin-dependent diabetes mellitus) results from total or near-complete destruction of the insulin-producing beta cells. This is the most common type of diabetes in dogs. As the name implies, dogs with this type of diabetes require insulin injections to stabilize blood sugar.

In type II diabetes mellitus (sometimes called non-insulin-dependent diabetes mellitus), some insulin-producing cells remain, but the amount of insulin produced is insufficient, there is a delayed response in secreting it, or the tissues of the dog's body are relatively insulin resistant. Type II diabetes may occur in older obese dogs. Humans with this form can often be treated with an oral drug that stimulates the remaining functional cells to produce or release insulin in an adequate amount to normalize blood sugar. Unfortunately, dogs do not respond well to these oral medications and usually need some insulin to control their disease.

Type III diabetes results from insulin resistance caused by other hormones and can be due to pregnancy or hormone-secreting tumors.

Insulin is an endogenous peptide hormone produced by beta cells of the pancreatic islets; it is considered to be the main anabolic hormone of the body. Insulin is the mainstay of therapy for diabetic dogs, and a conservative approach to insulin therapy is crucial. Most diabetic dogs require twice-daily dosing with lente or NPH insulin to adequately control their clinical signs. The current standard of care is twice daily insulin injections by the owner along with frequent veterinarian visits and disposable diagnostics that are expensive, time consuming and inconvenient for the owners of these animals.

What is needed are new therapies for the treatment of diabetes in canines.

### SUMMARY OF THE INVENTION

Viral vectors encoding insulin proteins adapted for use in canines are provided herein. In one aspect, a viral vector comprising a nucleic acid comprising a polynucleotide sequence encoding a canine insulin polypeptide is provided. In one embodiment, the vector is an adeno-associated viral vector. In another embodiment, the canine insulin polypeptide comprises a signal peptide and proinsulin polypeptide. In one embodiment, the signal peptide is a heterologous sequence. In another embodiment, the signal peptide is an insulin signal peptide. In another embodiment, the signal peptide comprises a canine IL2 signal peptide or a canine insulin signal peptide.

In one embodiment, the viral vector includes an AAV capsid, and a vector genome packaged in the AAV capsid, said vector genome comprising AAV inverted terminal repeats (ITRs), the polynucleotide sequence encoding the canine insulin polypeptide, and regulatory sequences which direct expression of the polypeptide.

In another embodiment, the viral vector includes an AAV capsid, and a vector genome packaged in the AAV capsid, said vector genome comprising AAV inverted terminal repeats (ITRs), the polynucleotide sequence encoding the canine insulin polypeptide, and regulatory sequences which direct insertion of the polynucleotide sequence encoding the polypeptide to the genome of a host cell.

In another aspect, a pharmaceutical composition suitable for use in treating a metabolic disease in a canine is provided. The pharmaceutical composition includes an aqueous liquid and a viral vector as described herein. according to any of claims 1 to 17.

In another aspect, a viral vector or pharmaceutical composition as described herein is provided for use in a method for treating a canine subject having a metabolic disease, optionally diabetes.

In another aspect, a viral vector or pharmaceutical composition as described herein is provided for use in the manufacture of a medicament for treating a canine subject having a metabolic disease, optionally diabetes.

In another aspect, a method of treating a canine subject having a metabolic disease is provided. The method includes comprising administering to the canine subject an effective amount of the viral vector or the pharmaceutical composition as described herein.

Other aspects and advantages of the invention will be readily apparent from the following detailed description of the invention.

### BRIEF DESCRIPTION OF THE DRAWINGS

FIG. 1 is a schematic showing the processing of insulin from preproinsulin to proinsulin to active insulin.

FIG. 2 is a schematic showing vector design, as discussed in Example 1.

FIG. 3 shows in vitro expression in HEK293 cells as described in Example 2.

FIG. 4A and FIG. 4B show body weight (FIG. 4A) and % body weight increase (FIG. 4B) in the mice discussed in FIG. 3.

FIG. 5A-FIG. 5C show blood glucose as measured by glucometer. The limit of the glucometer was 500 mg/dL. FIG. 5B and FIG. 5C are blood glucose traces for individual mice for cIns.2-1(N) and cIns.2-1(IL2) groups. X indicates the mouse died at D50.

FIG. 6 is a graph showing blood glucose as measured by a colorimetric detection kit. Colorimetric glucose assay was employed to determine blood glucose more accurately at serum samples at study week 0, 4, and 8.

FIG. 7 is a graph showing canine insulin levels at day 58 as determined by ELISA.

FIG. 8 is a plasmid map of pAAV.CB7.CI.cIns.2-1(N).rBG.

FIG. 9 is a plasmid map of pAAV.CB7.CI.cIns.2-1(IL2).rBG.

FIG. 10 is an alignment of the native canine insulin amino acid sequence (SEQ ID NO: 14; middle), the IL2.2-1 variant (SEQ ID NO: 4; top), and the N.2-1 variant (SEQ ID NO: 2; bottom).

FIG. 11A and FIG. 11B show blood glucose levels (FIG. 11A) and body weights (FIG. 11B) for STZ NOD-SCID mice administered mice AAVrh91.CB7.CI.cIns.2-1(N).rBG vector and PBS-administered STZ NOD-SCID and vehicle-administered NOD-SCID controls.

FIG. 12 shows insulin dose over 62 days for individual dogs in the group administered a high dose (1×10¹² gc/kg) of AAVrh91.CB7.CI.cIns.2-1(N).rBG.

FIG. 13 shows average insulin dose over 62 days for dogs administered a high (1×10¹² gc/kg) and low dose (1×10¹¹ gc/kg) of AAVrh91.CB7.CI.cIns.2-1(N).rBG.

### DETAILED DESCRIPTION OF THE INVENTION

Canine insulin proteins and expression constructs have been developed for use in canine animals. The term canine refers to any of species found in the Canidae family that among others includes domestic dogs, wolves, and foxes. In a preferred embodiment, the subject is a domestic dog, also known as Canis lupus familiaris or Canis familiaris.

Delivery of these constructs to subjects in need thereof via a number of routes, and particularly by expression in vivo mediated by a recombinant vector, such as a rAAV vector, is described. Also provided are methods of using these constructs in regimens for treating type 1 diabetes, type 2 diabetes, or metabolic syndrome in a veterinary subject in need thereof and increasing the level of insulin in a subject. In addition, methods are provided for enhancing the activity of insulin in a subject. Also provided are methods for inducing weight loss in a veterinary subject in need thereof.

Insulin is involved in regulation of glucose utilization in the body. Inability of the body to synthesize insulin or cells resistance to insulin leads to a condition called diabetes mellitus which is characterized by chronic hyperglycemia. Preproinsulin is transcribed as a 110 amino acid chain. Removal of the signal peptide produces proinsulin. Formation of disulfide bonds between the A- & B-chain components, and removal of the intervening C-chain, produces a biologically active Insulin molecule comprising 51 amino acids, less than half of the original translation product.

As used herein the term “insulin” refers to insulin or a functional fragment thereof, including proinsulin and preproinsulin, and amino-acid sequence variants of insulin or functional fragments thereof. The disclosure provides proteins comprising canine insulin, as well as polynucleotides and vectors encoding such proteins. In some embodiments, the insulin protein comprises a polynucleotide sequence encoding a polypeptide comprising (a) a leader sequence comprising a secretion signal peptide, and (b) a proinsulin polypeptide. In one embodiment, the protein comprises a canine IL2 leader sequence, and a canine proinsulin. In another embodiment, the protein comprises a canine insulin leader, and a canine proinsulin. The amino acid sequence of native canine insulin is shown in SEQ ID NO: 14.

In some embodiments, canine insulin includes variants which may include up to about 10% variation from an insulin nucleic acid or amino acid sequence described herein or known in the art, which retain the function of the wild-type sequence. As used herein, by “retain function” it is meant that the nucleic acid or amino acid functions in the same way as the wild type sequence, although not necessarily at the same level of expression or activity. For example, in one embodiment, a functional variant has increased expression or activity as compared to the wild type sequence. In another embodiment, the functional variant has decreased expression or activity as compared to the wild type sequence. In one embodiment, the functional variant has 10%, 20%, 30%, 40%, 50%, 60%, 70%, 80%, 90% or greater increase or decrease in expression or activity as compared to the wild type sequence.

In one embodiment, the insulin protein comprises a leader sequence, which may comprise a secretion signal peptide. As used herein, the term “leader sequence” refers to any N-terminal sequence of a polypeptide. In one embodiment, the canine insulin protein described herein comprises a leader, or signal sequence, and proinsulin. The leader sequence is, in one embodiment, a native sequence, or canine insulin, leader. In another embodiment, the leader sequence is a heterologous sequence, i.e., derived from another protein than canine insulin.

In one embodiment, the leader is a canine IL-2 sequence. In one embodiment, the IL-2 leader has the sequence shown in SEQ ID NO: 16: MYKMQLLSCIALTLVLVANS, or a functional variant thereof having at most 1, 2, or 3 amino acid substitutions.

In another embodiment, the leader is the native canine insulin sequence. In one embodiment, the canine leader has the sequence shown in SEQ ID NO: 17 MALWMRLLPLLALLALWAPAPTRA, or a functional variant thereof having at most 1, 2, or 3 amino acid substitutions.

The leader sequence may be derived from the same species for which administration is ultimately intended, i.e., a canine animal. As used herein, the terms “derived” or “derived from” mean the sequence or protein is sourced from a specific subject species or shares the same sequence as a protein or sequence sourced from a specific subject species. For example, a leader sequence which is “derived from” a canine, shares the same sequence (or a variant thereof, as defined herein) as the same leader sequence as expressed in a canine. However, the specified nucleic acid or amino acid need not actually be sourced from a canine. Various techniques are known in the art which are able to produce a desired sequence, including mutagenesis of a similar protein (e.g., a homolog) or artificial production of a nucleic acid or amino acid sequence. The “derived” nucleic acid or amino acid retains the function of the same nucleic acid or amino acid in the species from which it is “derived”, regardless of actual source of the derived sequence.

The term “amino acid substitution” and its synonyms are intended to encompass modification of an amino acid sequence by replacement of an amino acid with another, substituting, amino acid. The substitution may be a conservative substitution. It may also be a non-conservative substitution. The term conservative, in referring to two amino acids, is intended to mean that the amino acids share a common property recognized by one of skill in the art. For example, amino acids having hydrophobic nonacidic side chains, amino acids having hydrophobic acidic side chains, amino acids having hydrophilic nonacidic side chains, amino acids having hydrophilic acidic side chains, and amino acids having hydrophilic basic side chains. Common properties may also be amino acids having hydrophobic side chains, amino acids having aliphatic hydrophobic side chains, amino acids having aromatic hydrophobic side chains, amino acids with polar neutral side chains, amino acids with electrically charged side chains, amino acids with electrically charged acidic side chains, and amino acids with electrically charged basic side chains. Both naturally occurring and non-naturally occurring amino acids are known in the art and may be used as substituting amino acids in embodiments. Methods for replacing an amino acid are well known to the skilled in the art and include, but are not limited to, mutations of the nucleotide sequence encoding the amino acid sequence. Reference to “one or more” herein is intended to encompass the individual embodiments of, for example, 1, 2, 3, 4, 5, 6, or more.

The canine insulin protein also includes a proinsulin sequence/polypeptide. In one embodiment, the proinsulin sequence is the native canine proinsulin sequence shown in SEQ

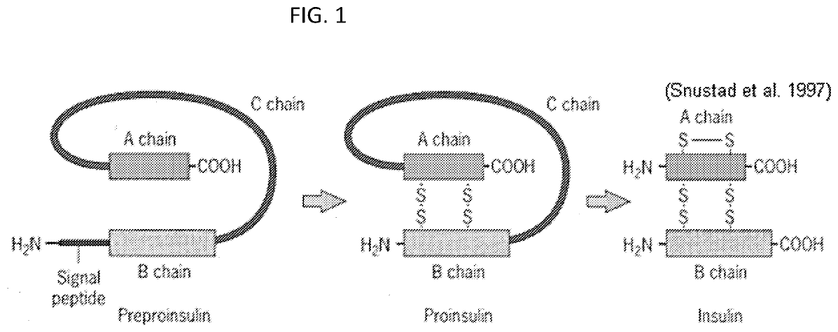

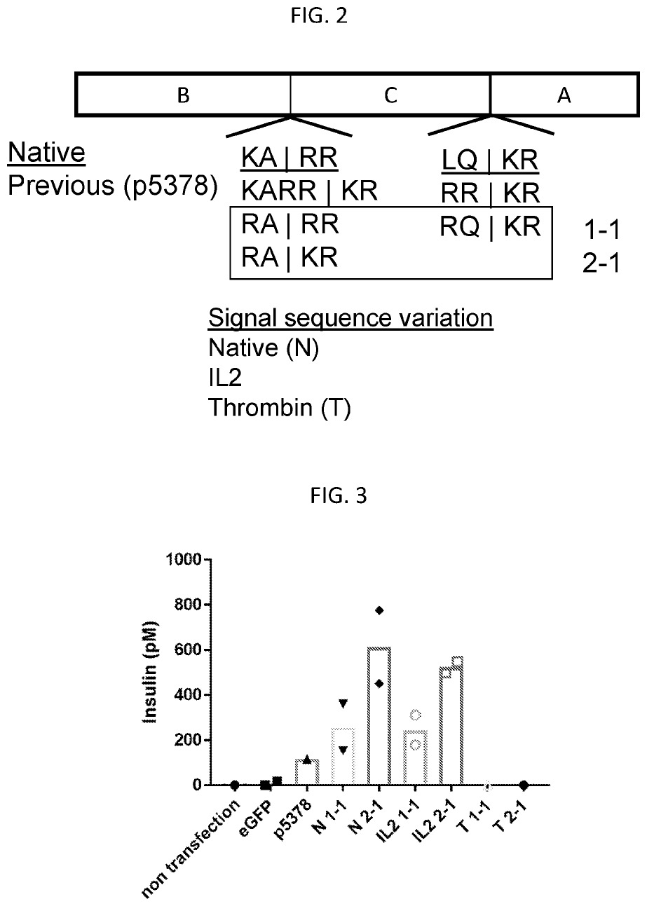

ID NO: 19:
FVNQHLCGSHLVEALYLVCGERGFFYTPKARRKREDLQVRDVELAGAPGEGGLQPL ALEGARRKRGIVEQCCTSICSLYQLENYCN. The proinsulin sequence, in one embodiment, contains one or more mutations as compared to the native sequence. These mutations are, in some embodiments, are in the cleavage sites between the B/C chains and C/A chains. See FIG. 1. In one embodiment, one or more of the cleavage sites are mutated to include a furin cleavage site. See, FIG. 2. In one embodiment, the proinsulin sequence has a 53-KARR-56 [symbol] 53-RAKR-56 mutation. In another embodiment, the proinsulin sequence has a 86-RRKR-89 [symbol] 86-RQKR-89 mutation. In another embodiment, the proinsulin sequence has both 53-KARR-56 [symbol] 53-RAKR-56 and 86-RRKR-89 [symbol] 86-RQKR-89 mutations. In one embodiment, the proinsulin sequence is SEQ ID NO: 18:
FVNQHLCGSHLVEALYLVCGERGFFYTPRAKREVEDLQVRDVELAGAPGEGGLQPL ALEGARQKRGIVEQCCTSICSLYQLENYCN (also termed 2-1), or a sequence having 1, 2 or 3 amino acid substitutions. In another embodiment, the canine proinsulin comprises SEQ ID NO: 18, or a functional variant thereof having at least 90%, at least 95%, at least 96%, at least 97%, at least 98%, at least 99%, or at least 99.5% identity with SEQ ID NO: 18.

In one embodiment, the canine insulin polypeptide comprises MALWMRLLPLLALLALWAPAPTRAFVNQHLCGSHLVEALYLVCGERGFFYTPRAK REVEDLQVRDVELAGAPGEGGLQPLALEGARQKRGIVEQCCTSICSLYQLENYCN (SEQ ID NO: 2) or a functional variant thereof having at most 1, 2, or 3 amino acid substitutions. In another embodiment, the canine insulin polypeptide comprises SEQ ID NO: 2, or a functional variant thereof having at least 90%, at least 95%, at least 96%, at least 97%, at least 98%, at least 99%, or at least 99.5% identity with SEQ ID NO: 2.

In another embodiment, the canine insulin polypeptide comprises MYKMQLLSCIALTLVLVANSFVNQHLCGSHLVEALYLVCGERGFFYTPRAKREVED LQVRDVELAGAPGEGGLQPLALEGARQKRGIVEQCCTSICSLYQLENYCN (SEQ ID NO: 4) or a functional variant thereof having at most 1, 2, or 3 amino acid substitutions. In another embodiment, the canine insulin polypeptide comprises SEQ ID NO: 2, or a functional variant thereof having at least 90%, at least 95%, at least 96%, at least 97%, at least 98%, at least 99%, or at least 99.5% identity with SEQ ID NO: 4.

In another embodiment, the canine insulin polypeptide comprises MYKMQLLSCIALTLVLVANSFVNQHLCGSHLVEALYLVCGERGFFYTPKARRKRED LQVRDVELAGAPGEGGLQPLALEGARRKRGIVEQCCTSICSLYQLENYCN (SEQ ID NO: 20) or a functional variant thereof having at most 1, 2, or 3 amino acid substitutions. In another embodiment, the canine insulin polypeptide comprises SEQ ID NO: 20, or a functional variant thereof having at least 90%, at least 95%, at least 96%, at least 97%, at least 98%, at least 99%, or at least 99.5% identity with SEQ ID NO: 20.

In addition to the leaders, proinsulin, and insulin polypeptides provided herein, nucleic acid sequences (used interchangeably with “polynucleotides”) encoding these polypeptides are provided. In one embodiment, a nucleic acid sequence is provided which encodes for the insulin polypeptide described herein. In some embodiments, this may include any nucleic acid sequence which encodes the insulin sequence of SEQ ID NO: 2. In another embodiment, this includes any nucleic acid which includes the insulin sequence of SEQ ID NO: 4. In another embodiment, this includes any nucleic acid which includes the insulin sequence of SEQ ID NO: 14. In yet another embodiment, this includes any nucleic acid which includes the insulin sequence of SEQ ID NO: 20.

In one embodiment, the sequence encoding the insulin protein is SEQ ID NO: 1 or a sequence at least 75%, at least 85%, at least 90%, at least 95%, at least 98%, or at least 99% identical thereto.

SEQ ID NO: 1
atggctctgtggatgagactgctgccactgctggctctgctcgcactgt
gggctccagctcctacaagagccttcgtgaaccagcacctgtgcggctc
tcatctggtggaagccctgtatctcgtgtgcggcgagaggggctttttc
tacacccctcgggccaagagagaggtcgaggatctgcaagtgcgcgacg
ttgaactggctggtgctcctggcgaaggtggactgcaacctctggctct
ggaaggcgccagacagaaaaggggcatcgtggaacagtgctgcaccagc
atctgcagcctgtaccagctggaaaactactgcaactgatga

In one embodiment, the sequence encoding the insulin protein is SEQ ID NO: 3 or a sequence at least 75%, at least 85%, at least 90%, at least 95%, at least 98%, or at least 99% identical thereto.

SEQ ID NO: 3
atgtacaagatgcagctgctgagctgtatcgccctgacactggtgctgg
tggccaacagcttcgtgaaccagcacctgtgcggaagccatctggtgga
agccctgtatctcgtgtgtggcgagaggggcttcttctacacccctcgg
gccaagagagaggtcgaggatctgcaagtgcgcgacgttgaactggctg
gtgctcctggcgaaggtggactgcaacctctggctctggaaggcgccag
acagaaaaggggcatcgtggaacagtgctgcaccagcatctgcagcctg
taccagctggaaaactactgcaactgatga

When a variant or fragment of the insulin sequence is desired, the coding sequences for these peptides may be generated using site-directed mutagenesis of the wild-type nucleic acid sequence. Alternatively or additionally, web-based or commercially available computer programs, as well as service-based companies may be used to back translate the amino acids sequences to nucleic acid coding sequences, including both RNA and/or cDNA. See, e.g., backtranseq by EMBOSS; Gene Infinity; and/or ExPasy. In one embodiment, the RNA and/or cDNA coding sequences are designed for optimal expression in the subject species for which administration is ultimately intended, i.e., a canine.

The coding sequences may be designed for optimal expression using codon optimization. Codon-optimized coding regions can be designed by various different methods. This optimization may be performed using methods which are available on-line, published methods, or a company which provides codon optimizing services. One codon optimizing method is described, e.g., in International Patent Application Pub. No. WO 2015/012924, which is incorporated by reference herein. Briefly, the nucleic acid sequence encoding the product is modified with synonymous codon sequences. Suitably, the entire length of the open reading frame (ORF) for the product is modified. However, in some embodiments, only a fragment of the ORF may be altered. By using one of these methods, one can apply the frequencies to any given polypeptide sequence, and produce a nucleic acid fragment of a codon-optimized coding region which encodes the polypeptide.

In another aspect, viral vectors comprising polynucleotides which encode the leaders, proinsulin, and insulin polypeptides as described herein, are provided. In certain embodiments of the viral vectors described herein, the viral vector is an adeno-associated virus (AAV) viral vector or recombinant AAV (rAAV). The term “recombinant AAV” or “rAAV” as used herein refers to naturally occurring adeno-associated viruses, adeno-associated viruses available to one of skill in the art and/or in light of the composition(s) and method(s) described herein, as well as artificial AAVs. An adeno-associated virus (AAV) viral vector is an AAV DNase-resistant particle having an AAV protein capsid into which is packaged an expression cassette flanked by AAV inverted terminal repeat sequences (ITRs) (together referred to as the “vector genome”) for delivery to target cells. An AAV capsid is composed of 60 capsid (cap) protein subunits, VP1, VP2, and VP3, that are arranged in an icosahedral symmetry in a ratio of approximately 1:1:10 to 1:1:20, depending upon the selected AAV. Various AAVs may be selected as sources for capsids of AAV viral vectors as identified above. In one embodiment, the AAV capsid is an AAVrh91 capsid or variant thereof. In certain embodiments, the capsid protein is designated by a number or a combination of numbers and letters following the term “AAV” in the name of the rAAV vector. Unless otherwise specified, the AAV capsid, ITRs, and other selected AAV components described herein, may be readily selected from among any AAV, including, without limitation, the AAVs identified as AAV1, AAV2, AAV3, AAV4, AAV5, AAV6, AAV7, AAV8, AAV9, AAVrh10, AAVhu37, AAVrh32.33, AAVAnc80, AAV10, AAV11, AAV12, AAVrh8, AAVrh74, AAV-DJ8, AAV-DJ, AAVhu37, AAVrh64R1, and AAVhu68. See, e.g., US Published Patent Application No. 2007-0036760-A1; US Published Patent Application No. 2009-0197338-A1; EP 1310571. See also, WO 2003/042397 (AAV7 and other simian AAV), U.S. Pat. Nos. 7,790,449 and 7,282,199 (AAV8), WO 2005/033321 and U.S. Pat. No. 7,906,111 (AAV9), and WO 2006/110689, and WO 2003/042397 (rh10), WO 2005/033321, WO 2018/160582 (AAVhu68), which are incorporated herein by reference. Other suitable AAVs may include, without limitation, AAVrh90 [PCT/US20/30273, filed Apr. 28, 2020], AAVrh91 [PCT/US20/30266, filed Apr. 28, 2020], AAVrh92, AAVrh93, AAVrh91.93 [PCT/US20/30281, filed Apr. 28, 2020], which are incorporated by reference herein. Other suitable AAV include AAV3B variants which are described in International Patent Application No. PCT/US20/56511, filed Oct. 20, 2020, describing AAV3B.AR2.01, AAV3B.AR2.02, AAV3B.AR2.03, AAV3B.AR2.04, AAV3B.AR2.05, AAV3B.AR2.06, AAV3B.AR2.07, AAV3B.AR2.08, AAV3B.AR2.10, AAV3B.AR2.11, AAV3B.AR2.12, AAV3B.AR2.13, AAV3B.AR2.14, AAV3B.AR2.15, AAV3B.AR2.16, or AAV3B.AR2.17, which are incorporated herein by reference. These documents also describe other AAV capsids which may be selected for generating rAAV and are incorporated by reference. Among the AAVs isolated or engineered from human or non-human primates (NHP) and well characterized, human AAV2 is the first AAV that was developed as a gene transfer vector; it has been widely used for efficient gene transfer experiments in different target tissues and animal models.

As used herein, relating to AAV, the term “variant” means any AAV sequence which is derived from a known AAV sequence, including those with a conservative amino acid replacement, and those sharing at least 90%, at least 95%, at least 97%, at least 99% or greater sequence identity over the amino acid or nucleic acid sequence. In another embodiment, the AAV capsid includes variants which may include up to about 10% variation from any described or known AAV capsid sequence. That is, the AAV capsid shares about 90% identity to about 99.9% identity, about 95% to about 99% identity or about 97% to about 98% identity to an AAV capsid provided herein and/or known in the art. In one embodiment, the AAV capsid shares at least 95% identity with an AAV capsid. When determining the percent identity of an AAV capsid, the comparison may be made over any of the variable proteins (e.g., vp1, vp2, or vp3).

In one embodiment, the viral vector is an rAAV having the capsid of AAVrh91 or a functional variant thereof. In one embodiment, the viral vector is an rAAV having the capsid of AAV3.AR.2.12 or a functional variant thereof In one embodiment, the viral vector is an rAAV having the capsid of AAV8 or a functional variant thereof In one embodiment, the viral vector is an rAAV having a capsid selected from AAV9, AAVrh64R1, AAVhu37, or AAVrh10.

In one aspect, a recombinant AAV (rAAV) is provided. The rAAV includes an AAV capsid from adeno-associated virus rh91, and a vector genome packaged in the AAV capsid, said vector genome comprising AAV inverted terminal repeats (ITRs), a coding sequence for the canine insulin of SEQ ID NO: 2, and regulatory sequences which direct expression of the canine insulin. In another embodiment, the rAAV includes an AAV capsid from adeno-associated virus rh91, and a vector genome packaged in the AAV capsid, said vector genome comprising AAV inverted terminal repeats (ITRs), a coding sequence for the canine insulin of SEQ ID NO: 4, and regulatory sequences which direct expression of the canine insulin. In another embodiment, the rAAV includes an AAV capsid from adeno-associated virus rh91, and a vector genome packaged in the AAV capsid, said vector genome comprising AAV inverted terminal repeats (ITRs), a coding sequence for the canine insulin of SEQ ID NO: 20, and regulatory sequences which direct expression of the canine insulin.

In one embodiment, the rAAV is an scAAV. The abbreviation “sc” refers to self-complementary. “Self-complementary AAV” refers a plasmid or vector having an expression cassette in which a coding region carried by a recombinant AAV nucleic acid sequence has been designed to form an intra-molecular double-stranded DNA template. Upon infection, rather than waiting for cell mediated synthesis of the second strand, the two complementary halves of scAAV will associate to form one double stranded DNA (dsDNA) unit that is ready for immediate replication and transcription. See, e.g., D M McCarty et al, “Self-complementary recombinant adeno-associated virus (scAAV) vectors promote efficient transduction independently of DNA synthesis”, Gene Therapy, (August 2001), Vol 8, Number 16, Pages 1248-1254. Self-complementary AAVs are described in, e.g., U.S. Pat. Nos. 6,596,535; 7,125,717; and 7,456,683, each of which is incorporated herein by reference in its entirety.

In one embodiment, the nucleic acid sequences encoding the insulin constructs described herein are engineered into any suitable genetic element, e.g., naked DNA, phage, transposon, cosmid, RNA molecule (e.g., mRNA), episome, etc., which transfers the insulin sequences carried thereon to a host cell, e.g., for generating nanoparticles carrying DNA or RNA, viral vectors in a packaging host cell and/or for delivery to a host cell in a subject. In one embodiment, the genetic element is a plasmid. The selected genetic element may be delivered by any suitable method, including transfection, electroporation, liposome delivery, membrane fusion techniques, high velocity DNA-coated pellets, viral infection and protoplast fusion. The methods used to make such constructs are known to those with skill in nucleic acid manipulation and include genetic engineering, recombinant engineering, and synthetic techniques. See, e.g., Green and Sambrook, Molecular Cloning: A Laboratory Manual, Cold Spring Harbor Press, Cold Spring Harbor, NY (2012).

As used herein, an “expression cassette” refers to a nucleic acid molecule which comprises the insulin construct coding sequences (e.g., coding sequences for the insulin protein), promoter, and may include other regulatory sequences therefor, which cassette may be engineered into a genetic element and/or packaged into the capsid of a viral vector (e.g., a viral particle). Typically, such an expression cassette for generating a viral vector contains the insulin construct sequences described herein flanked by packaging signals of the viral genome and other expression control sequences such as those described herein. Any of the expression control sequences can be optimized for a specific species using techniques known in the art including, e.g., codon optimization, as described herein.

The expression cassette typically contains a promoter sequence as part of the expression control sequences. In one embodiment, a constitutive promoter is used. In the plasmids and vectors described herein, a CB7 promoter is used. CB7 is a chicken β-actin promoter with cytomegalovirus enhancer elements. In one embodiment, the CB7 promoter comprises SEQ ID NO: 6 and/or SEQ ID NO: 7. Alternatively, liver-specific promoters may be used, such as those listed in The Liver Specific Gene Promoter Database, Cold Spring Harbor (available online at rulai.schl.edu/LSPD) and including but not limited to alpha 1 anti-trypsin (A1AT); human albumin (Miyatake et al., J. Virol. 71:5124 32 (1997)), humA1b; hepatitis B virus core promoter (Sandig et al., Gene Ther. 3:1002 9 (1996)); or a TTR minimal enhancer/promoter, alpha-antitrypsin promoter, or liver-specific promoter (LSP) (Wu et al. Mol Ther. 16:280-289 (2008)). In one embodiment, the liver-specific promoter thyroxin binding globulin (TBG) is used. Other promoters, such as viral promoters, constitutive promoters, regulatable promoters (see, e.g., WO 2011/126808 and WO 2013/04943) or a promoter responsive to physiologic cues may be used may be utilized in the vectors described herein. In another embodiment, an inducible promoter is used. An example of an inducible promoter useful herein includes that described in US Provisional Patent Application No. 63/056,985, filed Jul. 27, 2020, which is incorporated herein by reference. Briefly, the inducible promoter comprises a promoter; an activation domain comprising a canine or feline transactivation domain and a FKBP12-rapamycin binding (FRB) domain of canine or feline FKBP12-rapamycin-associated protein (FRAP); a DNA binding domain comprising a zinc finger homeodomain (ZFHD) and one, two or three FK506 binding protein domain (FKBP) subunit genes; and at least 8 copies of the binding site for ZFHD (8XZFHD) followed by a minimal IL2 promoter. The presence of an effective amount of a rapamycin or a rapalog induces expression of the transgene in a host cell.

In addition to a promoter, an expression cassette and/or a vector may contain other appropriate transcription initiation, termination, enhancer sequences, efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. Examples of suitable polyA sequences include, e.g., rabbit beta globin, SV40, bovine growth hormone (bGH), and TK polyA. Examples of suitable enhancers include, e.g., the alpha fetoprotein enhancer, the TTR minimal promoter/enhancer, LSP (TH-binding globulin promoter/alpha1-microglobulin/bikunin enhancer), amongst others. In one embodiment, the polyA is a rabbit globin polyA. In one embodiment, the polyA has the sequence of SEQ ID NO: 9.

These control sequences are “operably linked” to the insulin construct sequences. As used herein, the term “operably linked” refers to both expression control sequences that are contiguous with the gene of interest and expression control sequences that act in trans or at a distance to control the gene of interest.

In one embodiment, a rAAV is provided which includes a 5′ ITR, CB7 promoter, chicken beta-actin intron, coding sequence for the protein of SEQ ID NO: 2, a rabbit globin poly A, and a 3′ ITR. In one embodiment, a rAAV is provided which includes a 5′ ITR, CB7 promoter, chicken beta-actin intron, coding sequence for the protein of SEQ ID NO: 4, a rabbit globin poly A, and a 3′ ITR. In one embodiment, a rAAV is provided which includes a ITR, CB7 promoter, chicken beta-actin intron, coding sequence for the protein of SEQ ID NO: 14, a rabbit globin poly A, and a 3′ ITR. In one embodiment, a rAAV is provided which includes a 5′ ITR, CB7 promoter, chicken beta-actin intron, coding sequence for the protein of SEQ ID NO: 20, a rabbit globin poly A, and a 3′ ITR.

The minimal sequences required to package the expression cassette into an AAV viral particle are the AAV 5′ and 3′ ITRs, which may be of the same AAV origin as the capsid, or of a different AAV origin (to produce an AAV pseudotype). In one embodiment, the ITR sequences from AAV2, or the deleted version thereof (AITR), are used for convenience and to accelerate regulatory approval. However, ITRs from other AAV sources may be selected. Preferably, the source of the ITRs is the same as the source of the Rep protein, which is provided in trans for production. Typically, an expression cassette for an AAV vector comprises an AAV 5′ ITR, the insulin fusion protein coding sequences and any regulatory sequences, and an AAV 3′ ITR. However, other configurations of these elements may be suitable. A shortened version of the 5′ ITR, termed AITR, has been described in which the D-sequence and terminal resolution site (trs) are deleted. In other embodiments, the full-length AAV 5′ and 3′ ITRs are used. Examples of suitable ITRs are provided in SEQ ID NOs: 5 and 10.

For packaging an expression cassette into virions, the ITRs are the only AAV components required in cis in the same construct as the gene. In one embodiment, the coding sequences for the replication (rep) and/or capsid (cap) are removed from the AAV genome and supplied in trans or by a packaging cell line in order to generate the AAV vector. For example, as described above, a pseudotyped AAV may contain ITRs from a source which differs from the source of the AAV capsid. In one embodiment, a chimeric AAV capsid may be utilized. Still other AAV components may be selected. Sources of such AAV sequences are described herein and may also be isolated or obtained from academic, commercial, or public sources (e.g., the American Type Culture Collection, Manassas, VA). The AAV sequences may be obtained through synthetic or other suitable means by reference to published sequences such as are available in the literature or in databases such as, e.g., GenBank, PubMed, or the like.

Methods for generating and isolating AAV viral vectors suitable for delivery to a subject are known in the art. See, e.g., U.S. Pat. Nos. 7,790,449; 7,282,199; WO 2003/042397; WO 2005/033321, WO 2006/110689; and U.S. Pat. No. 7,588,772 B2]. In a one system, a producer cell line is transiently transfected with a construct that encodes the transgene flanked by ITRs and a construct(s) that encodes rep and cap. In a second system, a packaging cell line that stably supplies rep and cap is transiently transfected with a construct encoding the transgene flanked by ITRs. In each of these systems, AAV virions are produced in response to infection with helper adenovirus or herpesvirus, requiring the separation of the rAAVs from contaminating virus. More recently, systems have been developed that do not require infection with helper virus to recover the AAV - the required helper functions (i.e., adenovirus E1, E2a, VA, and E4 or herpesvirus UL5, UL8, UL52, and UL29, and herpesvirus polymerase) are also supplied, in trans, by the system. In these newer systems, the helper functions can be supplied by transient transfection of the cells with constructs that encode the required helper functions, or the cells can be engineered to stably contain genes encoding the helper functions, the expression of which can be controlled at the transcriptional or posttranscriptional level. In yet another system, the transgene flanked by ITRs and rep/cap genes are introduced into insect cells by infection with baculovirus-based vectors. For reviews on these production systems, see generally, e.g., Zhang et al., 2009, “Adenovirus-adeno-associated virus hybrid for large-scale recombinant adeno-associated virus production,” Human Gene Therapy 20:922-929, the contents of each of which is incorporated herein by reference in its entirety. Methods of making and using these and other AAV production systems are also described in the following U.S. patents, the contents of each of which is incorporated herein by reference in its entirety: U.S. Pat. Nos. 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065. See generally, e.g., Grieger & Samulski, 2005, “Adeno-associated virus as a gene therapy vector: Vector development, production and clinical applications,” Adv. Biochem. Engin/Biotechnol. 99:119-145; Buning et al., 2008, “Recent developments in adeno-associated virus vector technology,” J. Gene Med. 10:717-733; and the references cited below, each of which is incorporated herein by reference in its entirety. The methods used to construct any embodiment of this invention are known to those with skill in nucleic acid manipulation and include genetic engineering, recombinant engineering, and synthetic techniques. See, e.g., Green and Sambrook et al, Molecular Cloning: A Laboratory Manual, Cold Spring Harbor Press, Cold Spring Harbor, NY (2012). Similarly, methods of generating rAAV virions are well known and the selection of a suitable method is not a limitation on the present invention. See, e.g., K. Fisher et al, (1993) J. Virol., 70:520-532 and U.S. Pat. No. 5,478,745.

The rAAV described herein comprise a selected capsid with a vector genome packaged inside. The vector genome (or rAAV genome) comprises 5′ and 3′ AAV inverted terminal repeats (ITRs), the polynucleotide sequence encoding the insulin protein, and regulatory sequences which direct insertion of the polynucleotide sequence encoding the insulin protein to the genome of a host cell. In one embodiment, the vector genome is the sequence shown in SEQ ID NO: 11 or a sequence sharing at least 90%, at least 95%, or at least 99% identity therewith. In one embodiment, the vector genome is the sequence shown in SEQ ID NO: 12 or a sequence sharing at least 90%, at least 95%, or at least 99% identity therewith. In one embodiment, the vector genome is the sequence shown in SEQ ID NO: 15 or a sequence sharing at least 90%, at least 95%, or at least 99% identity therewith.

Optionally, the insulin constructs described herein may be delivered via viral vectors other than rAAV. Such other viral vectors may include any virus suitable for gene therapy may be used, including but not limited to adenovirus; herpes virus; lentivirus; retrovirus; etc. Suitably, where one of these other vectors is generated, it is produced as a replication-defective viral vector.

A “replication-defective virus” or “viral vector” refers to a synthetic or artificial viral particle in which an expression cassette containing a gene of interest is packaged in a viral capsid or envelope, where any viral genomic sequences also packaged within the viral capsid or envelope are replication-deficient; i.e., they cannot generate progeny virions but retain the ability to infect target cells. In one embodiment, the genome of the viral vector does not include genes encoding the enzymes required to replicate (the genome can be engineered to be “gutless”—containing only the transgene of interest flanked by the signals required for amplification and packaging of the artificial genome), but these genes may be supplied during production. Therefore, it is deemed safe for use in gene therapy since replication and infection by progeny virions cannot occur except in the presence of the viral enzyme required for replication.

Also provided are compositions which include the viral vector constructs described herein. The pharmaceutical compositions described herein are designed for delivery to canine subjects in need thereof by any suitable route or a combination of different routes. Direct delivery to the liver (optionally via intravenous, via the hepatic artery, or by transplant), direct delivery to the pancreas, oral, inhalation, intranasal, intratracheal, intraarterial, intraocular, intravenous, intramuscular, subcutaneous, intradermal, and other parental routes of administration. The viral vectors described herein may be delivered in a single composition or multiple compositions. Optionally, two or more different AAV may be delivered, or multiple viruses [see, e.g., WO 2011/126808 and WO 2013/049493]. In another embodiment, multiple viruses may contain different replication-defective viruses (e.g., AAV and adenovirus). In one embodiment, administration is intramuscular. In another embodiment, administration is intravenous.

The replication-defective viruses can be formulated with a physiologically acceptable carrier for use in gene transfer and gene therapy applications. In the case of AAV viral vectors, quantification of the genome copies (“GC” or “gc”) may be used as the measure of the dose contained in the formulation. Any method known in the art can be used to determine the genome copy (GC) number of the replication-defective virus compositions of the invention. One method for performing AAV GC number titration is as follows: Purified AAV vector samples are first treated with DNase to eliminate un-encapsidated AAV genome DNA or contaminating plasmid DNA from the production process. The nuclease resistant particles are then subjected to heat treatment to release the genome from the capsid. The released genomes are then quantitated by real-time PCR using primer/probe sets targeting specific region of the viral genome (usually poly A signal). Another suitable method for determining genome copies is the quantitative-PCR (qPCR), particularly the optimized qPCR or digital droplet PCR [Lock Martin, et al, Human Gene Therapy Methods. April 2014, 25(2): 115-125. doi:10.1089/hgtb.2013.131, published online ahead of editing Dec. 13, 2013].

Also, the replication-defective virus compositions can be formulated in dosage units to contain an amount of replication-defective virus that is in the range of about 1.0×10⁹ GC to about 1.0×10¹⁵ GC. In another embodiment, this amount of viral genome may be delivered in split doses. In one embodiment, the dose is about 1.0×10¹⁰ GC to about 3.0×10¹³ GC for an average canine subject of about 5-10 kg. In another embodiment, the dose about 1×10⁹ GC. For example, the dose of AAV virus may be about 1×10¹⁰ GC, 1×10¹¹ GC, about 5×10¹¹ GC, about 1×10¹² GC, about 5×10¹² GC, or about 1×10¹³ GC. In another embodiment, the dosage is about 1.0×10⁹ GC/kg to about 3.0×10¹³ GC/kg for a canine subject. In another embodiment, the dose about 1×10⁹ GC/kg. For example, the dose of AAV virus may be about 1×10¹⁰ GC/kg, 1×10¹¹ GC/kg, about 5×10¹¹ GC/kg, about 1×10¹² GC/kg, about 5×10¹² GC/kg, or about 1×10¹³ GC/kg. In one embodiment, the constructs may be delivered in volumes from 111 μL to about 100 mL for a veterinary subject. See, e.g., Diehl et al, J. Applied Toxicology, 21:15-23 (2001) for a discussion of good practices for administration of substances to various veterinary animals. This document is incorporated herein by reference. As used herein, the term “dosage” can refer to the total dosage delivered to the subject in the course of treatment, or the amount delivered in a single (of multiple) administration.

The above-described recombinant vectors may be delivered to host cells according to published methods. The rAAV, preferably suspended in a physiologically compatible carrier, may be administered to a desired subject including a canine. Suitable carriers may be readily selected by one of skill in the art in view of the indication for which the transfer virus is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present invention.

Optionally, the compositions of the invention may contain, in addition to the rAAV and/or variants and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

The viral vectors and other constructs described herein may be used in preparing a medicament for delivering an insulin protein construct to a subject in need thereof, supplying insulin having an increased half-life to a subject, and/or for treating type I diabetes, type II diabetes, or metabolic syndrome in a subject. Thus, in another aspect a method of treating diabetes is provided. The method includes administering a composition as described herein to a canine subject in need thereof. In one embodiment, the composition includes a viral vector containing an insulin protein expression cassette, as described herein.

In another embodiment, a method for treating type 2 diabetes in a canine is provided. The method includes administering a viral vector comprising a nucleic acid molecule comprising a sequence encoding an insulin protein as described herein.

In another embodiment, a method for treating type 1 diabetes in a canine is provided. The method includes administering a viral vector comprising a nucleic acid molecule comprising a sequence encoding an insulin protein as described herein.

In another aspect, a method of treating a metabolic disease in a canine is provided. The method includes administering a composition as described herein to a canine subject in need thereof In one embodiment, the composition includes a viral vector containing an insulin protein expression cassette, as described herein. In one embodiment, the metabolic disease is Type I diabetes. In one embodiment, the metabolic disease is Type II diabetes. In one embodiment, the metabolic disease is metabolic syndrome.

A course of treatment may optionally involve repeat administration of the same viral vector (e.g., an AAVrh91 vector) or a different viral vector (e.g., an AAVrh91 and an AAV3B.AR2.12). Still other combinations may be selected using the viral vectors described herein. Optionally, the composition described herein may be combined in a regimen involving other diabetic drugs or protein-based therapies (including e.g., insulin analogues, insulin, oral antihyperglycemic drugs (sulfonylureas, biguanides, thiazolidinediones, and alpha-glucosidase inhibitors). Optionally, the composition described herein may be combined in a regimen involving lifestyle changes including dietary and exercise regimens.

As used herein the terms “insulin construct”, “insulin expression construct” and synonyms include the insulin sequence as described herein in combination with a leader (whether native or heterologous). The terms “insulin construct”, “insulin expression construct” and synonyms can be used to refer to the nucleic acid sequences encoding the insulin protein or the expression products thereof.

The terms “percent (%) identity”, “sequence identity”, “percent sequence identity”, or “percent identical” in the context of nucleic acid sequences refers to the bases in the two sequences which are the same when aligned for correspondence. The length of sequence identity comparison may be over the full-length of the genome, the full-length of a gene coding sequence, or a fragment of at least about 100 to 150 nucleotides, or as desired. However, identity among smaller fragments, e.g., of at least about nine nucleotides, usually at least about 20 to 24 nucleotides, at least about 28 to 32 nucleotides, at least about 36 or more nucleotides, may also be desired. Multiple sequence alignment programs are also available for nucleic acid sequences. Examples of such programs include, “Clustal W”, “CAP Sequence Assembly”, “BLAST”, “MAP”, and “MEME”, which are accessible through Web Servers on the internet. Other sources for such programs are known to those of skill in the art. Alternatively, Vector NTI utilities are also used. There are also a number of algorithms known in the art that can be used to measure nucleotide sequence identity, including those contained in the programs described above. As another example, polynucleotide sequences can be compared using Fasta, a program in GCG Version 6.1. Fasta provides alignments and percent sequence identity of the regions of the best overlap between the query and search sequences. For instance, percent sequence identity between nucleic acid sequences can be determined using Fasta with its default parameters (a word size of 6 and the NOPAM factor for the scoring matrix) as provided in GCG Version 6.1, herein incorporated by reference.

The terms “percent (%) identity”, “sequence identity”, “percent sequence identity”, or “percent identical” in the context of amino acid sequences refers to the residues in the two sequences which are the same when aligned for correspondence. Percent identity may be readily determined for amino acid sequences over the full-length of a protein, polypeptide, about 70 amino acids to about 100 amino acids, or a peptide fragment thereof or the corresponding nucleic acid sequence coding sequencers. A suitable amino acid fragment may be at least about 8 amino acids in length, and may be up to about 150 amino acids. Generally, when referring to “identity”, “homology”, or “similarity” between two different sequences, “identity”, “homology” or “similarity” is determined in reference to “aligned” sequences. “Aligned” sequences or “alignments” refer to multiple nucleic acid sequences or protein (amino acids) sequences, often containing corrections for missing or additional bases or amino acids as compared to a reference sequence. Alignments are performed using any of a variety of publicly or commercially available Multiple Sequence Alignment Programs. Sequence alignment programs are available for amino acid sequences, e.g., the “Clustal X”, “MAP”, “PIMA”, “MSA”, “BLOCKMAKER”, “MEME”, and “Match-Box” programs. Generally, any of these programs are used at default settings, although one of skill in the art can alter these settings as needed. Alternatively, one of skill in the art can utilize another algorithm or computer program which provides at least the level of identity or alignment as that provided by the referenced algorithms and programs. See, e.g., J. D. Thomson et al, Nucl. Acids. Res., “A comprehensive comparison of multiple sequence alignments”, 27(13):2682-2690 (1999).

It is to be noted that the term “a” or “an” refers to one or more. As such, the terms “a” (or “an”), “one or more,” and “at least one” are used interchangeably herein.

The words “comprise”, “comprises”, and “comprising” are to be interpreted inclusively rather than exclusively. The words “consist”, “consisting”, and its variants, are to be interpreted exclusively, rather than inclusively. While various embodiments in the specification are presented using “comprising” language, under other circumstances, a related embodiment is also intended to be interpreted and described using “consisting of” or “consisting essentially of” language.

As used herein, the term “about” means a variability of 10% from the reference given, unless otherwise specified.

The term “regulation” or variations thereof as used herein refers to the ability of a composition to inhibit one or more components of a biological pathway.

As used herein, “disease”, “disorder” and “condition” are used interchangeably, to indicate an abnormal state in a subject.

Unless defined otherwise in this specification, technical and scientific terms used herein have the same meaning as commonly understood by one of ordinary skill in the art and by reference to published texts, which provide one skilled in the art with a general guide to many of the terms used in the present application.

A reference to “one embodiment” or “another embodiment” in describing an embodiment does not imply that the referenced embodiment is mutually exclusive with another embodiment (e.g., an embodiment described before the referenced embodiment), unless expressly specified otherwise.

The following examples are illustrative only and are not intended to limit the present invention.

### EXAMPLE 1—CONSTRUCTION OF INSULIN VECTORS

Canine pre-proinsulin contains two cleavage sites. Vectors were constructed containing canine insulin sequences as follows. In the construct name, N refers to native signal sequence, IL2 refers to IL2 signal sequence, and T refers to thrombin signal sequence. A schematic of these amendments is shown in FIG. 2.
Native canine insulin (cIns(Native)) (SEQ ID NO: 14);
Canine insulin, where the native sequence has been mutated to include an insertion/mutation at the first cleavage site, and 1 mutation in the second site (p5378);
Canine insulin, where the native sequence has been mutated to include 2 furin cleavage sites, with 1 mutation in the first site, and 1 mutation in the second site (cIns.N.1-1);
Canine insulin, where the native sequence has been mutated to include 2 furin cleavage sites, with 1 mutation in the first site, and 1 mutation in the second site, and where the native signal sequence has been swapped with the IL2 signal sequence (cIns.IL2.1-1);
Canine insulin, where the native sequence has been mutated to include 2 furin cleavage sites, with 2 mutations in the first site, and 1 mutation in the second site (cIns.N.2-1) (SEQ ID NO: 2);
Canine insulin, where the native sequence has been mutated to include 2 furin cleavage sites, with 2 mutations in the first site, and 1 mutation in the second site, and where the native signal sequence has been swapped with the IL2 signal sequence (cIns.IL2.2-1) (SEQ ID NO: 4).
Canine insulin, where the native sequence has been mutated to include 2 furin cleavage sites, with 1 mutation in the first site, and 1 mutation in the second site, and where the native signal sequence has been swapped with the thrombin signal sequence (cIns.T.1-1);
Canine insulin, where the native sequence has been mutated to include 2 furin cleavage sites, with 2 mutations in the first site, and 1 mutation in the second site, and where the native signal sequence has been swapped with the thrombin signal sequence (cIns.T.2-1).

The protein sequences were back translated, and engineered for optimal expression in canines, followed by addition of a Kozak consensus sequence, stop codon, and cloning sites. The sequences were produced, and cloned into an expression vector containing a hybrid cytomegalovirus enhancer/chicken b-actin promoter. The expression construct was flanked by AAV2 ITRs.

### EXAMPLE 2—IN VITRO EXPRESSION

The purified plasmids for the constructs were transfected into triplicate wells of a 6 well plate of 90% confluent HEK 293 cells using lipofectamine 2000 according to the manufacturer's instructions. Supernatant was harvested 48 hours after transfection and insulin was measured using ELISA. The expression of the constructs is shown in FIG. 3. The 2-1 constructs using IL2 or native signal sequence performed best in vitro.

### EXAMPLE 3—EXPRESSION IN STZ-NOD SCID MICE

The following constructs were packaged in an AAVrh91 vector by triple transfection and iodixanol gradient purification, as previously described:
AAVrh91.CB7.CI.cIns.2-1(N).rBG, with native insulin signal; and
AAVrh91.CB7.CI.cIns.2-1(IL2).rBG, with canine IL2 signal In addition, the following construct was packaged into an AAV8 vector:
AAV8.CB7.CI.clns.rBG, with native insulin signal

| Group | N | Vector | Dose (GC/mouse) |
|---|---|---|---|
| 1 | 3 | PBS | N/A |
| 2 | 3 | AAV8.CB7.CI.cIns.rBG | 1.00 × 10¹¹ |
| 3 | 4 | AAVrh91.CB7.CI.cIns.2-1(N).rBG | 1.00 × 10¹¹ |
| 4 | 4 | AAVrh91.CB7.CI.cIns.2-1(IL2).rBG | 1.00 × 10¹¹ |

STZ-NOD SCID mice were administered vector (1×10¹¹ GC/mouse) via IM injection. Twice per week, fasting blood glucose was taken, where food was removed from the cages 6 hours prior to testing. Once per month, fasting serum insulin was tested. One mouse from group 4 was euthanized at day 50 due to hypoglycemia. Three mice from group 1 were euthanized at day 84 due to seizure-like activity and low BCS.

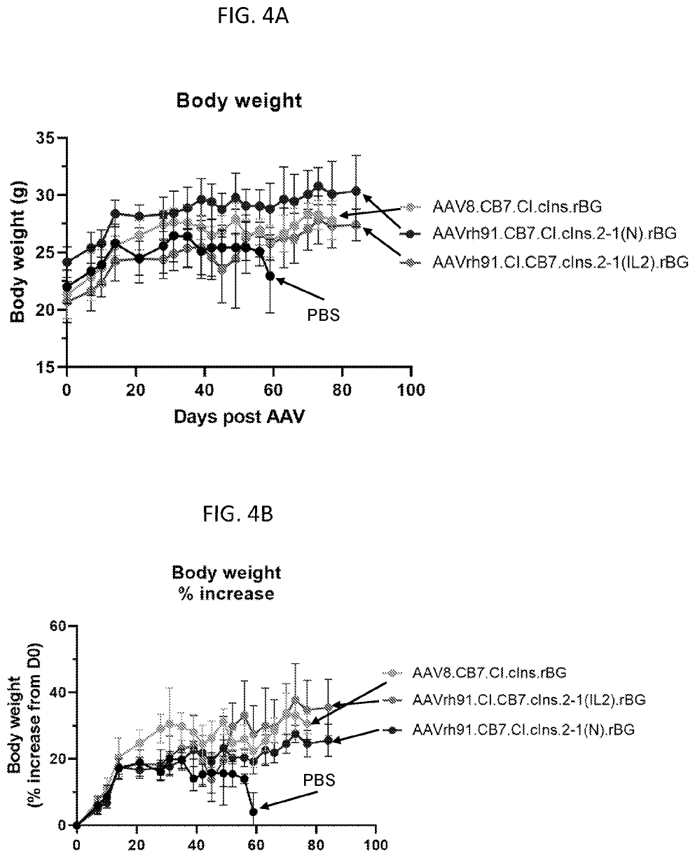

Body weight and body weight increases of the mice are shown in FIG. 4A and FIG. 4B. Significant changes in body weight were not observed.

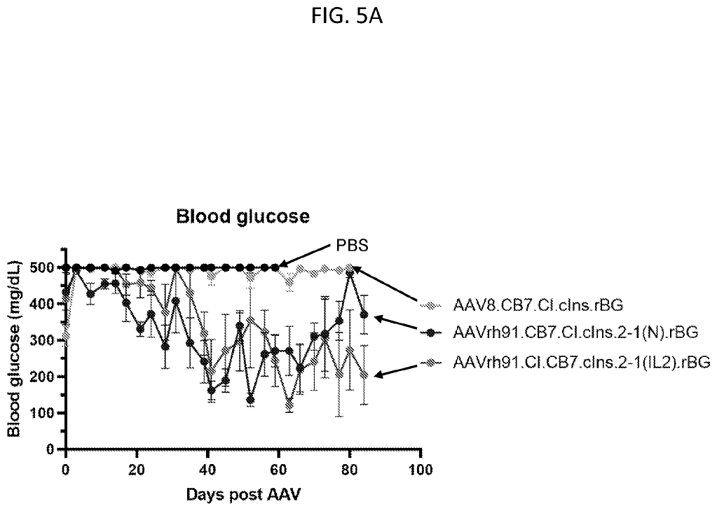

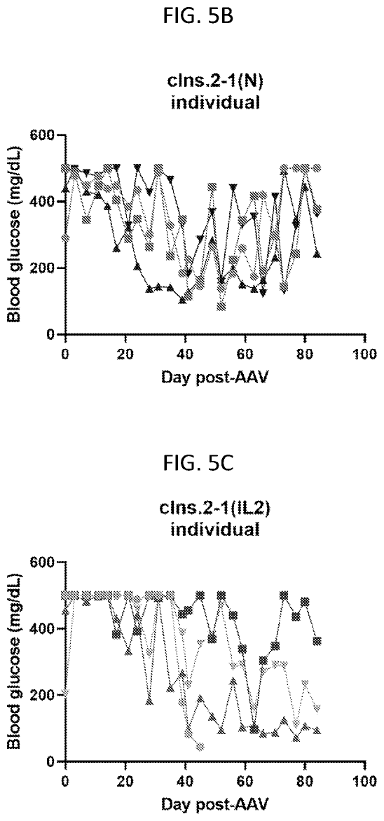

Fasting blood glucose was measured by glucometer, which had an upper limit of 500 mg/dL. Animals receiving the AAVrh91 vectors had significant reduction in blood glucose levels, while AAV8 vector was not effective (FIG. 5A). FIG. 5B and FIG. 5C are blood glucose traces for individual mice for clns.2-1(N) and clns.2-1(IL2) groups. X indicates the mouse died at D50.

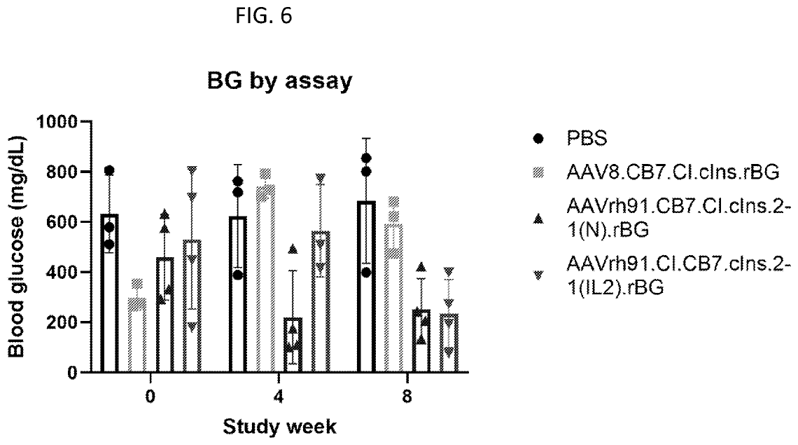

Colorimetric glucose assay was employed to determine blood glucose more accurately at serum samples at study week 0, 4, and 8 (FIG. 6). Again, significant reductions in blood glucose were observed with the AAVrh91 vectors.

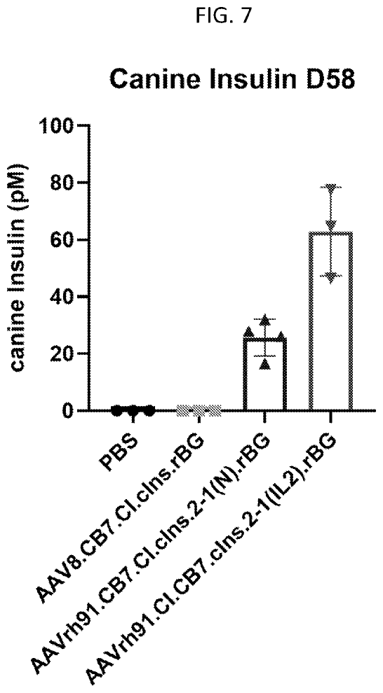

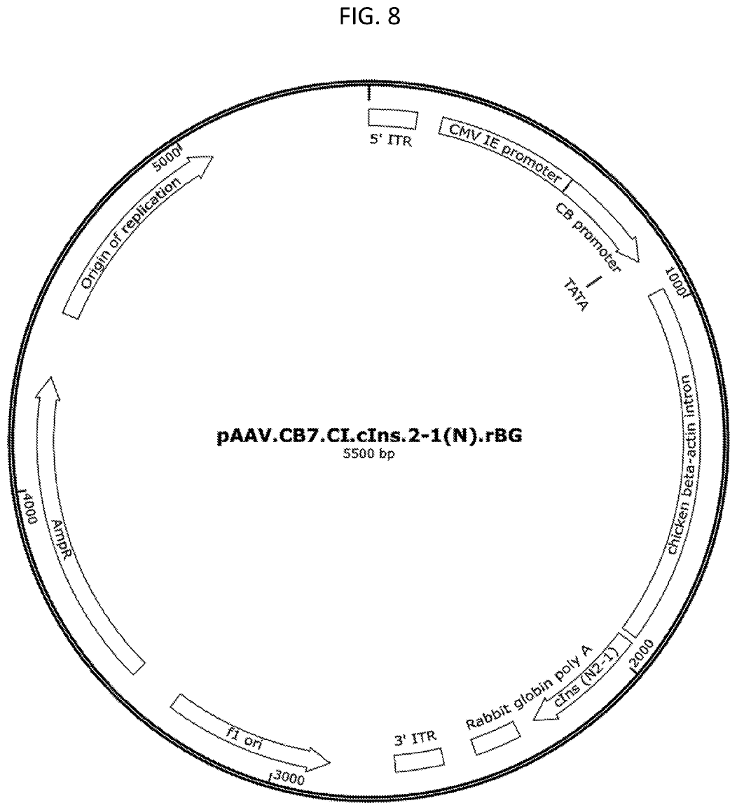

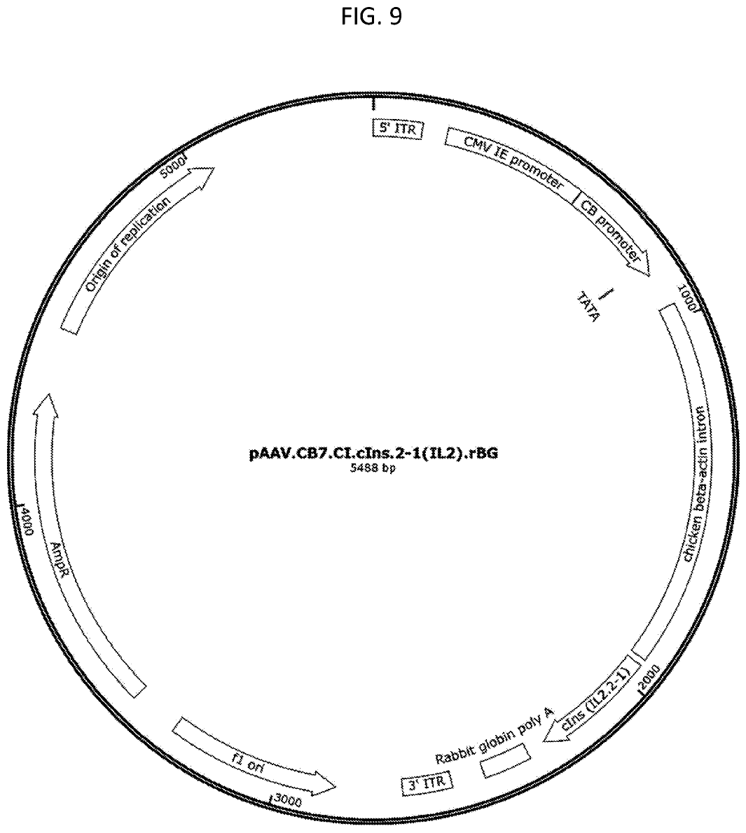

A canine insulin ELISA was performed on day 58 mouse serum (FIG. 7). Significant levels of canine insulin were seen with AAVrh91 vectors, with IL2 signal sequence construct providing highest levels.

An additional study was performed with a larger number of animals to evaluate delivery of the AAVrh91.CB7.CI.cIns.2-1(N).rBG vector. As outlined in the table below, seven STZ NOD-SCID mice were administered 1.00×10¹¹ GC of vector. PBS-administered STZ NOD-SCID mice and vehicle-administered NOD-SCID mice server as controls.

| Group | 1 | 2 | 3 |
|---|---|---|---|
| NOD-SCID males | 7 | 10 | 7 |
| (N) | | | |
| STZ | STZ | Vehicle | STZ |
| Vector (IM) | PBS | NA | AAVrh91.CB7.CI.cIns.2-1(N).rBG |
| Dose (GC/mouse) | NA | NA | 1.00E+11 |

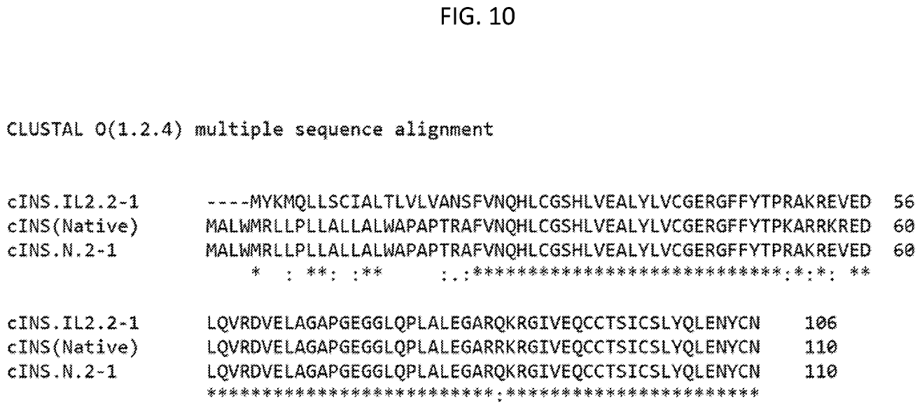

Fasting blood glucose and body weights were measured as described above for 56 days (FIG. 10A and FIG. 10B). Significant reductions in blood glucose level were observed in STZ NOD-SCID mice administered the AAVrh91.CB7.CI.cIns.2-1(N).rBG vector.

### EXAMPLE 4—DELIVERY OF AAV CANINE INSULIN IN AN STZ DOG MODEL

A study was performed to evaluate the efficacy of a single IM administration of an AAVrh91 vector expressing canine insulin (AAVrh91.CB7.CI.cIns.2-1(N).rBG) for adequately maintaining blood glucose levels in diabetic dogs at a low dose (1×10¹¹ gc/kg, N=3) and a high dose (1×10¹² gc/kg, N=3). This was a non-randomized, non-blinded, laboratory study including six Beagle dogs with chemically induced diabetes mellitus.

On the first day of baseline (Day−2), subjects were acclimated to housing conditions and a series of health screening measures including veterinary examinations, serum fructosamine analysis as well as hematology, clinical chemistry analysis, and urinalysis were performed, deeming all subjects suitable to continue onto the trial. In addition, 3× daily blood glucose (BG) measurements via glucometer and 2× daily clinical observations were initiated. On Day−1, subjects were acclimated to placement of continuous glucose monitors (CGM) and 3× daily interstitial fluid (ISF) glucose value capture via CGMs was initiated.

During acclimation, animals were maintained on a previously established diabetic treatment regimen of twice daily Caninsulin (MSD Animal Health) administration following AM and PM feed offerings. In the evening on Day−1, Caninsulin doses were withheld to provide a washout prior to baseline blood collections for insulin/anti-insulin samples taken on Day 0.

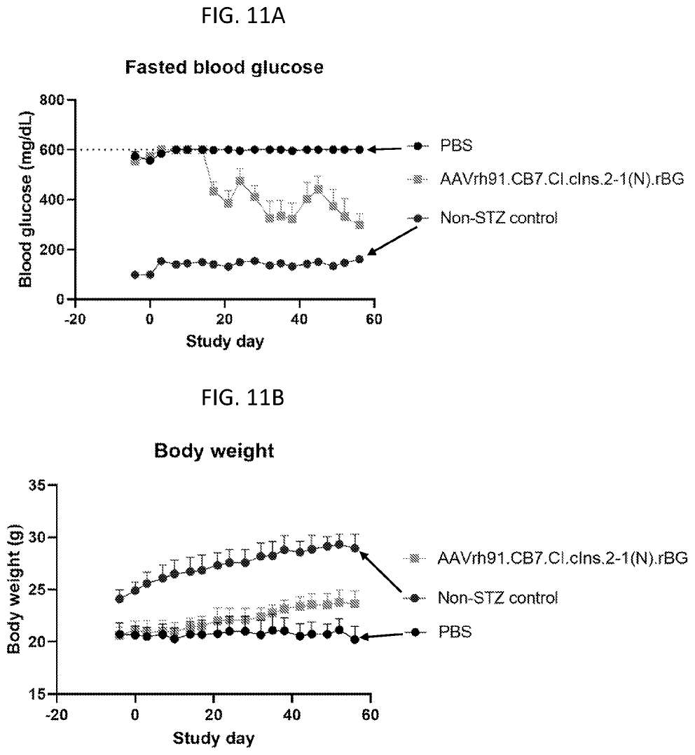

Over the course of study, in addition to the daily procedures initiated at baseline, blood collections for fructosamine analysis, hematology and clinical chemistry were performed every 14 days until Day 42 and once more on Day 63. Blood collections for determining endogenous insulin and anti-insulin antibodies were performed throughout the study on Days 0, 5, 7, 10, 14, 17, 21, 24, 28, 31, 35, 38, 42, 45, 49, 56, and 63. Insulin was withheld at the PM timepoint on the day prior to scheduled blood collection procedures. Collections for urinalysis occurred on Days 14, 28, 42 and 63, and veterinary examinations were performed every 14 days. Animal body weights were measured every 7 days (FIG. 11B).

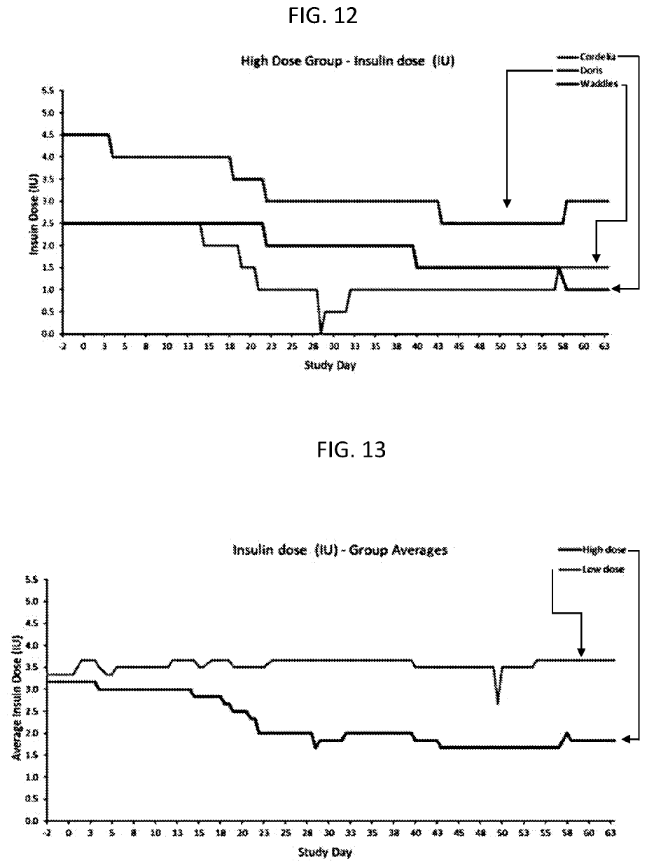

Required supplementary insulin (Caninsulin) dose levels and serum insulin levels were used to evaluate the efficacy of the test product at the two doses tested in individual subjects. All dogs that received a high dose (1×10¹² gc/kg) of the vector required substantial and sustained decreases in Caninsulin dose over the 63 days following administration of the product (FIG. 12). No dogs in the low dose (1×10¹¹ gc/kg) group experienced appreciable or sustained decreases in their maintenance insulin dose.

On average, dogs in the high dose group experienced noteworthy increases in serum insulin levels, which is consistent with clinical responses seen regarding reduction in exogenous insulin dose requirements (FIG. 12). In the low dose group dogs, the serum insulin levels increased only small amounts which were reflected in the inability to reduce exogenous insulin doses (FIG. 13).

Based on the results of this study, delivery of the vector at a dose of 1×10¹² gc/kg was shown to improve diabetic control in dogs with chemically induced diabetes for up to 63 days, resulting in a decreased need for exogenous insulin over this time (FIG. 12). Administration of 1×10¹¹ gc/kg did not result in significant diabetic control.

### SEQUENCE LISTING FREE TEXT

The following information is provided for sequences containing free text under numeric identifier <223>.

| SEQ ID NO: | |
|---|---|
| (containing free text) | Free text under <223> |
| 1 | <223> Constructed Sequence |
| 2 | <223> Constructed sequence |
| 3 | <223> Constructed sequence |
| 4 | <223> Constructed sequence |
| 6 | <223> CMV IE promoter |
| 7 | <223> CB promoter |
| 8 | <223> CBA intron |
| 9 | <223> Rabbit beta globin poly A |
| 11 | <223> Constructed sequence |
| 12 | <223> Constructed sequence |
| 15 | <223> Constructed sequence |
| 18 | <223> Constructed sequence |
| 20 | <223> Constructed sequence |

All publications cited in this specification, are incorporated herein by reference. US Provisional Patent Application No. 63/109,620, filed Nov. 4, 2020, is incorporated by reference. Similarly, the SEQ ID NOs which are referenced herein and which appear in the Sequence Listing filed herewith, labeled “UPN-20-9293PCT_Seq-Listing_ST25”, are incorporated by reference. While the invention has been described with reference to particular embodiments, it will be appreciated that modifications can be made without departing from the spirit of the invention. Such modifications are intended to fall within the scope of the appended claims.

1. A viral vector comprising a nucleic acid comprising a polynucleotide sequence encoding a canine insulin polypeptide.

2. The viral vector according to claim 1, wherein the vector is an adeno-associated viral vector.

3. The viral vector according to claim 1, wherein the canine insulin polypeptide comprises a signal peptide and proinsulin polypeptide.

4. The viral vector according to claim 3, wherein the signal peptide is a heterologous sequence.

5. The viral vector according to claim 3, wherein the signal peptide is an insulin signal peptide.

6. The viral vector according to claim 5, wherein the signal peptide comprises a canine IL2 signal peptide or a canine insulin signal peptide.

7. The viral vector according to claim 3, wherein the signal peptide comprises the sequence of MYKMQLLSCIALTLVLVANS (SEQ ID NO: 16) or a functional variant thereof having at most 1, 2, or 3 amino acid substitutions; or the sequence of MALWMRLLPLLALLALWAPAPTRA (SEQ ID NO: 17) or a functional variant thereof having at most 1, 2, or 3 amino acid substitutions.

8. The viral vector according to claim 1, wherein the canine insulin is a variant having a mutation at one or more cleavage sites.

9. The viral vector according to claim 3, wherein the canine proinsulin polypeptide comprises
FVNQHLCGSHLVEALYLVCGERGFFYTPRAKREVEDLQVRDVELAGAPGEGGL QPLALEGARQKRGIVEQCCTSICSLYQLENYCN (SEQ ID NO: 18) or a functional variant thereof having at most 1, 2, or 3 amino acid substitutions.

10. The viral vector according to claim 7, wherein the canine insulin polypeptide comprises
MALWMRLLPLLALLALWAPAPTRAFVNQHLCGSHLVEALYLVCGERGFFYTPR AKREVEDLQVRDVELAGAPGEGGLQPLALEGARQKRGIVEQCCTSICSLYQLENYCN (SEQ ID NO: 2) or a functional variant thereof having at most 1, 2, or 3 amino acid substitutions.

11. The viral vector according to claim 7, wherein the canine insulin polypeptide comprises
MYKMQLLSCIALTLVLVANSFVNQHLCGSHLVEALYLVCGERGFFYTPRAKREV EDLQVRDVELAGAPGEGGLQPLALEGARQKRGIVEQCCTSICSLYQLENYCN (SEQ ID NO: 4) or a functional variant thereof having at most 1, 2, or 3 amino acid substitutions.

12. The viral vector according to claim 7, wherein the canine insulin polypeptide comprises
MYKMQLLSCIALTLVLVANSFVNQHLCGSHLVEALYLVCGERGFFYTPKARRKR EDLQVRDVELAGAPGEGGLQPLALEGARRKRGIVEQCCTSICSLYQLENYCN (SEQ ID NO: 20) or a functional variant thereof having at most 1, 2, or 3 amino acid substitutions.

13. The viral vector according to claim 2, comprising:
(a) an AAV capsid, and
(b) a vector genome packaged in the AAV capsid, said vector genome comprising AAV inverted terminal repeats (ITRs), the polynucleotide sequence encoding the canine insulin polypeptide, and regulatory sequences which direct expression of the polypeptide.

14. The viral vector according to claim 2, comprising:
(a) an AAV capsid, and
(b) a vector genome packaged in the AAV capsid, said vector genome comprising AAV inverted terminal repeats (ITRs), the polynucleotide sequence encoding the canine insulin polypeptide, and regulatory sequences which direct insertion of the polynucleotide sequence encoding the polypeptide to the genome of a host cell.

15. The viral vector according to claim 2, wherein the viral vector is an rAAV having the capsid of AAVrh91, AAV1, AAV3B.AR2.12, AAV8, AAV9, AAVrh.64R1, AAVhu.37, or AAVrh.10 or a functional variant thereof.

16-17. (canceled)

18. A pharmaceutical composition suitable for use in treating a metabolic disease in a canine comprising an aqueous liquid and the viral vector according to claim 1.

19-21. (canceled)

22. A method of treating a canine subject having a metabolic disease, comprising administering to the canine subject an effective amount of the pharmaceutical composition according to claim 18.

23. The method according to claim 22, wherein the metabolic disease is diabetes.

24-25. (canceled)

26. The method according to claim 22, wherein the effective amount is administered intravenously or intramuscularly.

27. (canceled)

28. The method according to claim 22, wherein the effective amount is between 1×10⁹ GC/kg to 3×10¹³ GC/kg body mass of the rAAV.

29. (canceled)
